(19) United States
(12) Patent Application Publication
(10) Pub. No.: US 2007/0293763 A1
(43) Pub. Date: Dec. 20, 2007

(54) ULTRASONIC PROBE APPARATUS AND ULTRASONIC DIAGNOSING APPARATUS

(76) Inventor: Kiyoshi Matsumura, Tokyo (JP)

Correspondence Address: GENERAL ELECTRIC CO.;GLOBAL PATENT OPERATION, 187 Danbury Road, Suite 204, Wilton, CT, 06897-4122, US

(21) Appl. No.: 11/756,492

(22) Filed: May 31, 2007

(30) Foreign Application Priority Data
Jun. 15, 2006 (JP) 2006-165433

(51) Int. Cl. A61B 8/00

(52) U.S. Cl. 600/459

(57) ABSTRACT

To make it possible to check whether or not an ultrasonic probe apparatus has been normally connected to the ultrasonic diagnosing apparatus body by merely looking at the ultrasonic probe apparatus, a connector box of an ultrasonic probe apparatus is provided with an active state indicating LED and a sleeping state indicating LED. An indicator control unit receives an active flag setting signal from the ultrasonic diagnosing apparatus body and, if the switch of a locking mechanism is “on”, turns on the active state indicating LED. Further, it receives a sleep flag setting signal from the ultrasonic diagnosing apparatus body and, if the switch of the locking mechanism is “on”, turns on the sleeping state indicating LED.

### BACKGROUND OF THE INVENTION

The present invention relates to an ultrasonic probe apparatus and an ultrasonic diagnosing apparatus, and more particularly to an ultrasonic probe apparatus and an ultrasonic diagnosing apparatus which makes it possible to know, by merely looking at the ultrasonic probe apparatus, whether or not the ultrasonic probe apparatus is normally connected to the ultrasonic diagnosing apparatus body.

An ultrasonic diagnosing apparatus body provided with a probe connector unit on a plane in the same direction as an operation unit, and an ultrasonic probe apparatus provided with an information display unit for identifying the type of the ultrasonic probe apparatus by direct observation on the plane in the same direction as the operation unit of the ultrasonic diagnosing apparatus body (see Japanese Unexamined Patent Publication No. 2001-258881 for instance).

The conventional ultrasonic probe apparatus described above makes it possible to know, by merely looking at the display unit of the ultrasonic diagnosing apparatus body, whether or not the ultrasonic probe apparatus is normally connected to the ultrasonic diagnosing apparatus body.

However, it involves a problem that whether or not the ultrasonic probe apparatus is normally connected to the ultrasonic diagnosing apparatus body cannot be known by merely looking at the ultrasonic probe apparatus.

### SUMMARY OF THE INVENTION

Therefore, an object of the present invention is to provide an ultrasonic probe apparatus and an ultrasonic diagnosing apparatus which makes it possible to know, by merely looking at the ultrasonic probe, whether or not the ultrasonic probe is normally connected to the ultrasonic diagnosing apparatus body.

According to a first aspect of the invention, there is provided an ultrasonic probe apparatus comprising: a connector box having on a first face a connector unit to be connected to the probe connector unit of an ultrasonic diagnosing apparatus body and having on a second face an indicator unit which is lit when the connector unit is normally connected to the probe connector unit of the ultrasonic diagnosing apparatus body; and an ultrasonic probe connected to the connector box by a cable.

The ultrasonic probe apparatus according to the foregoing first aspect makes it possible to know, when the ultrasonic probe apparatus is connected to the ultrasonic diagnosing apparatus body, whether or not the connection has been accomplished normally by looking at the lit state of the indicator unit. Therefore, the operator need not stretch his or her body for looking at the display unit of the ultrasonic diagnosing apparatus body from the posture at the time the ultrasonic probe apparatus was connected to the ultrasonic diagnosing apparatus body, whereby the workload on the operator can be correspondingly reduced.

According to a second aspect of the invention, there is provided the ultrasonic probe apparatus according to the first aspect wherein the second face is on the reverse side to the first face.

The ultrasonic probe apparatus according to the foregoing second aspect, as the indicator unit comes into the operator's field of view when he or she connects the ultrasonic probe apparatus to the ultrasonic diagnosing apparatus body, the operator can almost automatically check the indicator unit.

According to a third aspect of the invention, there is provided the ultrasonic probe apparatus according to the foregoing first or second aspect wherein the indicator unit varies in the lit or unlit state according to whether the ultrasonic probe apparatus is active or sleeping.

When an ultrasonic probe apparatus connected to an ultrasonic diagnosing apparatus body is to be disconnected, the ultrasonic probe apparatus should not be active. Thus, it is necessary to make sure before disconnecting the ultrasonic probe apparatus that it is sleeping. Previously, this was done by looking at the display unit of the ultrasonic diagnosing apparatus body.

The ultrasonic probe apparatus according to the foregoing third aspect does not require the operator to stretch his or her body to look at the display unit of the ultrasonic diagnosing apparatus body from the posture at the time he or she tried to disconnect the ultrasonic probe apparatus from the ultrasonic diagnosing apparatus body, and the workload on the operator can be correspondingly reduced.

According to a fourth aspect of the invention, there is provided the ultrasonic probe apparatus according to the third aspect wherein the indicator unit varies in the color of light according to whether the ultrasonic probe apparatus is active or sleeping.

The ultrasonic probe apparatus according to the foregoing fourth aspect enables the operator to visually check whether the ultrasonic probe apparatus is active or sleeping by looking at the color of light of the indicator unit.

According to a fifth aspect of the invention, there is provided the ultrasonic probe apparatus according to the third or fourth aspect wherein the indicator unit varies in the luminous intensity of light according to whether the ultrasonic probe apparatus is active or sleeping.

The ultrasonic probe apparatus according to the foregoing fifth aspect enables the operator to visually check whether the ultrasonic probe apparatus is active or sleeping by looking at the luminance of the lit indicator unit.

According to a sixth aspect of the invention, there is provided the ultrasonic probe apparatus according to any of the third through fifth aspects wherein the indicator unit has separate light sources including one to be lit when the ultrasonic probe apparatus is active and the other to be lit when the ultrasonic probe apparatus is sleeping.

The ultrasonic probe apparatus according to the foregoing sixth aspect enables the operator to visually check whether the ultrasonic probe apparatus is active or sleeping by seeing which of the light sources is on.

According to a seventh aspect of the invention, there is provided the ultrasonic probe apparatus according to any of the first through sixth aspects wherein the indicator unit also serves as a name plate.

The ultrasonic probe apparatus according to the foregoing seventh aspect is suitable for use with a small connector because the area of the name plate showing the model number of the apparatus can be utilized.

According to an eighth aspect of the invention, there is provided the ultrasonic probe apparatus according to any of the first through seventh aspects wherein the connector box is provided with a locking mechanism which locks a state in which the connector unit is connected to the probe connector unit of the ultrasonic diagnosing apparatus body, and the indicator unit is not lit in a state in which the lock is undone.

The ultrasonic probe apparatus according to the foregoing eighth aspect enables the operator also to visually check whether or not the locking is normally accomplished.

According to a ninth aspect of the invention, there is provided an ultrasonic diagnosing apparatus comprising: an ultrasonic diagnosing apparatus body having a probe connector unit; a connector box having on a first face a connector unit to be connected to the probe connector unit and having on a second face an indicator unit which is lit when the connector unit is normally connected to the probe connector unit of the ultrasonic diagnosing apparatus body; and an ultrasonic probe connected to the connector box by a cable.

The ultrasonic diagnosing apparatus according to the foregoing ninth aspect makes it possible to know, when the ultrasonic probe apparatus is connected to the ultrasonic diagnosing apparatus body, whether or not the connection has been accomplished normally by looking at the lit state of the indicator unit. Therefore, the operator need not stretch his or her body for looking at the display unit of the ultrasonic diagnosing apparatus body, from the posture at the time the ultrasonic probe apparatus was connected to the ultrasonic diagnosing apparatus body, and the workload on the operator can be correspondingly reduced.

According to a tenth aspect of the invention, there is provided the ultrasonic diagnosing apparatus according to the ninth aspect wherein the second face is on the reverse side to the first face.

The ultrasonic probe apparatus according to the foregoing tenth aspect, as the indicator unit comes into the operator's field of view when he or she connects the ultrasonic probe apparatus to the ultrasonic diagnosing apparatus body, the operator can almost automatically check the indicator unit.

According to an eleventh aspect of the invention, there is provided the ultrasonic diagnosing apparatus according to the foregoing ninth or tenth aspect wherein the indicator unit varies in the lit or unlit state according to whether the ultrasonic probe apparatus is active or sleeping.

The ultrasonic probe apparatus according to the foregoing eleventh aspect does not require the operator to stretch his or her body to look at the display unit of the ultrasonic diagnosing apparatus body from the posture at the time he or she tried to disconnect the ultrasonic probe apparatus from the ultrasonic diagnosing apparatus body, and the workload on the operator can be correspondingly reduced.

According to a twelfth aspect of the invention, there is provided the ultrasonic diagnosing apparatus according to the foregoing eleventh aspect wherein the indicator unit varies in the color of light according to whether the ultrasonic probe apparatus is active or sleeping.

The ultrasonic probe apparatus according to the foregoing twelfth aspect enables the operator to visually check whether the ultrasonic probe apparatus is active or sleeping by looking at the color of light of the indicator unit.

According to a thirteenth aspect of the invention, there is provided the ultrasonic diagnosing apparatus according to the foregoing eleventh aspect wherein the indicator unit varies in the luminous intensity of light according to whether the ultrasonic probe apparatus is active or sleeping.

The ultrasonic probe apparatus according to the foregoing thirteenth aspect enables the operator to visually check whether the ultrasonic probe apparatus is active or sleeping by looking at the luminance of the lit indicator unit.

According to a fourteenth aspect of the invention, there is provided the ultrasonic diagnosing apparatus according to the foregoing eleventh or twelfth aspect wherein the indicator unit has separate light sources including one to be lit when the ultrasonic probe apparatus is active and the other to be lit when the ultrasonic probe apparatus is sleeping.

The ultrasonic probe apparatus according to the foregoing fourteenth aspect enables the operator to visually check whether the ultrasonic probe apparatus is active or sleeping by seeing which of the light sources is on.

According to a fifteenth aspect of the invention, there is provided the ultrasonic diagnosing apparatus according to any of the foregoing ninth through fourteenth aspects wherein the indicator unit also serves as a name plate.

The ultrasonic probe apparatus according to the foregoing fifteenth aspect is suitable for use with a small connector because the area of the name plate showing the model number of the apparatus can be utilized.

According to a sixteenth aspect of the invention, there is provided the ultrasonic diagnosing apparatus according to any of the foregoing ninth through fifteenth aspects wherein the connector box is provided with a locking mechanism which locks a state in which the connector unit is connected to the probe connector unit of the ultrasonic diagnosing apparatus body, and the indicator unit is not lit in a state in which the lock is undone.

The ultrasonic probe apparatus according to the foregoing sixteenth aspect enables the operator also to visually check whether or not the lock is normally accomplished.

The ultrasonic probe apparatus and the ultrasonic diagnosing apparatus according to the invention make it possible, when the ultrasonic probe apparatus is connected to the ultrasonic diagnosing apparatus body, to check whether or not the connection is normal by looking at the lit state of the indicator unit. Therefore, the operator need not stretch his or her body from the posture at the time the ultrasonic probe apparatus was connected to the ultrasonic diagnosing apparatus body to look at the display unit of the ultrasonic diagnosing apparatus body, and the workload on the operator can be correspondingly reduced.

The invention can be utilized in ultrasonic diagnosing apparatuses.

Further objects and advantages of the present invention will be apparent from the following description of the preferred embodiments of the invention as illustrated in the accompanying drawings.

### BRIEF DESCRIPTION OF THE DRAWINGS

FIG. 1 shows a perspective view of an ultrasonic diagnosing apparatus, which is Embodiment 1 of the invention.

FIG. 2 shows a front view of an ultrasonic probe apparatus pertaining to Embodiment 1.

FIG. 3 is a configurational block diagram showing functions of an ultrasonic probe apparatus pertaining to Embodiment 1.

FIG. 4 shows a front view of an ultrasonic probe apparatus pertaining to Embodiment 2 of the invention.

FIG. 5 is a configurational block diagram showing functions of an ultrasonic probe apparatus pertaining to Embodiment 3 of the invention.

FIG. 6 is a configurational block diagram showing functions of an ultrasonic probe apparatus pertaining to Embodiment 4 of the invention.

### DETAILED DESCRIPTION OF THE INVENTION

The present invention will be described in further detail below with reference to preferred embodiments thereof illustrated in the accompanying drawings. Incidentally, the invention is not limited to these embodiments.

### Embodiment 1

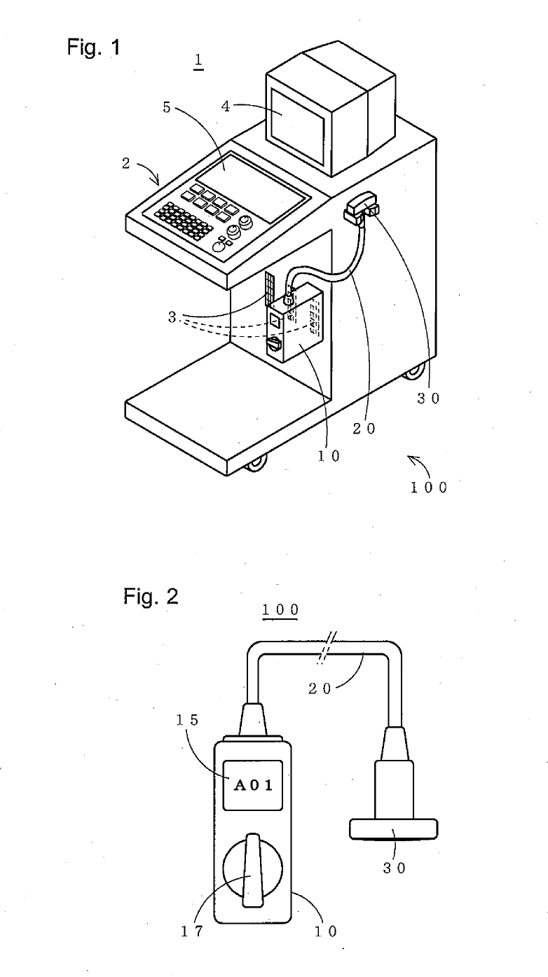

FIG. 1 shows a perspective view of an ultrasonic diagnosing apparatus, which is Embodiment 1 of the invention.

This ultrasonic diagnosing apparatus 1 is provided with an ultrasonic diagnosing apparatus body 2 and an the ultrasonic probe apparatus 100.

The ultrasonic diagnosing apparatus body 2 has three probe connector units 3 in the lower part of the front, a display unit 4 and a touch panel unit 5.

The ultrasonic probe apparatus 100 is provided with a connector box 10 and an ultrasonic probe 30 connected to the connector box 10 by a cable 20.

FIG. 2 shows an external view of the ultrasonic probe apparatus 100.

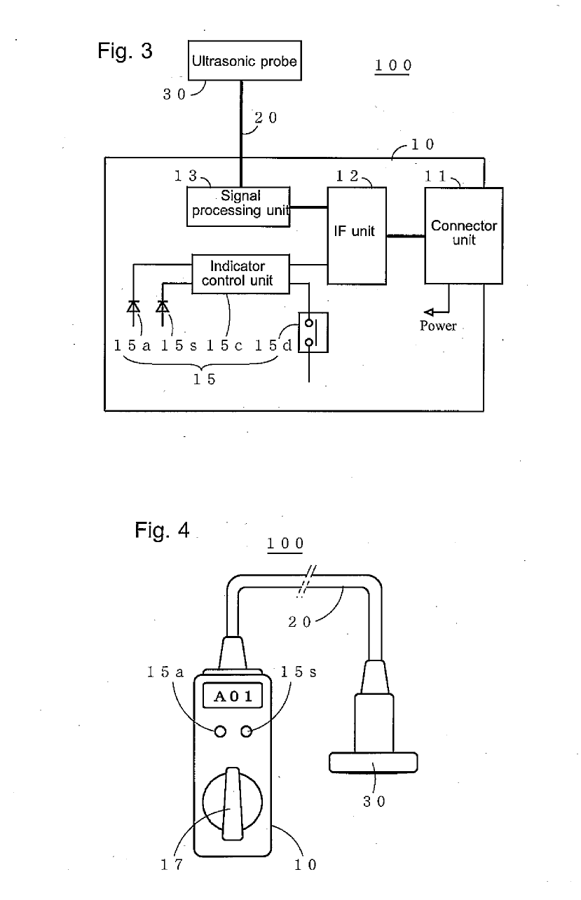

On the front face of the connector box 10, an indicator unit 15 and a lock lever 17 are disposed. On the rear face not illustrated, there is arranged a connector unit (denoted by 11 in FIG. 3), which is to be connected to the probe connector units 3 of the ultrasonic diagnosing apparatus body 2.

The indicator unit 15 also serves a name plate which shows model number “A01” of the ultrasonic probe apparatus 100.

The lock lever 17, when it is in an upright position as shown in FIG. 2, is not locked, and enables the connector unit (11 in FIG. 3) to be connected to or disconnected from the probe connector units 3 of the ultrasonic diagnosing apparatus body 2. By turning the lock lever 17 by 90° as shown in FIG. 1 in a state in which the connector unit (11 in FIG. 3) is connected to the probe connector units 3 of the ultrasonic diagnosing apparatus body 2, the connected state can be locked.

FIG. 3 is a configurational block diagram showing functions of the ultrasonic probe apparatus 100.

Power is supplied to the connector box 10 from the ultrasonic diagnosing apparatus body 2 via the connector unit 11.

An interface unit 12 transmits to a signal processing unit 13 a transmission control signal received from the ultrasonic diagnosing apparatus body 2 via the connector unit 11, and transmits to the ultrasonic diagnosing apparatus body 2 an echo signal received from the signal processing unit 13 via the connector unit 11.

The signal processing unit 13 drives an element of the ultrasonic probe 30 in accordance with the transmission control signal, and generates the echo signal based on an ultrasonic echo received by the element of the ultrasonic probe 30.

The indicator unit 15 comprises a green and bright active state indicating LED 15a, an orange and less bright sleeping state indicating LED 15s, a switch 15d which is turned “on” in a locked state, and an indicator control unit 15c which controls the turning on and off of the active state indicating LED 15a and the sleeping state indicating LED 15s.

The active state indicating LED 15a and the sleeping state indicating LED 15s are positioned inside a translucent name plate.

The indicator control unit 15c has a two-bit flag.

This flag is reset for “00”, which means that the power supply is on, when the power supply is started. It is turned to “10” by an active flag setting signal received from the ultrasonic diagnosing apparatus body 2 via the connector unit 11 and the interfacing unit 12. It is further turned to “01” by a sleep flag setting signal received from the ultrasonic diagnosing apparatus body 2 via the connector unit 11 and the interfacing unit 12.

The indicator control unit 15c turns on neither the active state indicating LED 15a nor the sleeping state indicating LED 15s if the flag is “00”. If the flag is “10” and the switch 15d is “on”, it turns on the active state indicating LED 15a. If the flag is “01” and the switch 15d is “on”, it turns on the sleeping state indicating LED 15s.

The ultrasonic probe apparatus 100 and the ultrasonic diagnosing apparatus 1 of Embodiment 1 provide the following advantages.

(1) When the ultrasonic probe apparatus 100 is connected to the ultrasonic diagnosing apparatus body 2, if the indicator unit 15 is lit in either green or orange, it indicates normal connection. If neither is lit, it indicates either failure to normally connect them or the occurrence of some trouble in the ultrasonic diagnosing apparatus body 2 and/or the ultrasonic probe apparatus 100.

(2) The operator, who leans forward when connecting the ultrasonic probe apparatus 100 to the ultrasonic diagnosing apparatus body 2, can look at the indicator unit 15 in that posture to see if the apparatus is active, sleeping or not normal. Therefore, the operator need not stretch himself or herself to see indications on the display unit 4 and the touch panel unit 5 of the ultrasonic diagnosing apparatus body 2, and is thereby relieved of some of the workload.

(3) As the indicator unit 15 comes into the operator's field of view when he or she connects the ultrasonic probe apparatus 100 to the ultrasonic diagnosing apparatus body 2, the operator can almost automatically check the indicator unit 15.

(4) The operator, who leans forward when disconnecting the ultrasonic probe apparatus 100 from the ultrasonic diagnosing apparatus body 2, can look at the indicator unit 15 in that posture to see the apparatus is not active but sleeping. Therefore, the operator need not stretch himself or herself to see indications on the display unit 4 and the touch panel unit 5 of the ultrasonic diagnosing apparatus body 2, and is thereby relieved of some of the workload.

(5) By seeing the lit color of the indicator unit 15, the operator can visually know whether the ultrasonic probe apparatus 100 is active or sleeping.

(6) By seeing the relative luminance of the lit indicator unit 15, the operator can also visually know whether the ultrasonic probe apparatus 100 is active or sleeping.

(7) The indicator unit 15 shares the area of the name plate, and this feature contributes to reducing the size of the connector box 10.

(8) By looking at the lit or unlit state of the indicator unit 15, the operator can also check whether or not the connection is normally locked.

### Embodiment 2

As shown in FIG. 4, the active state indicating LED 15a and the sleeping state indicating LED 15s may as well be arranged side by side in other area than the name plate.

### Embodiment 3

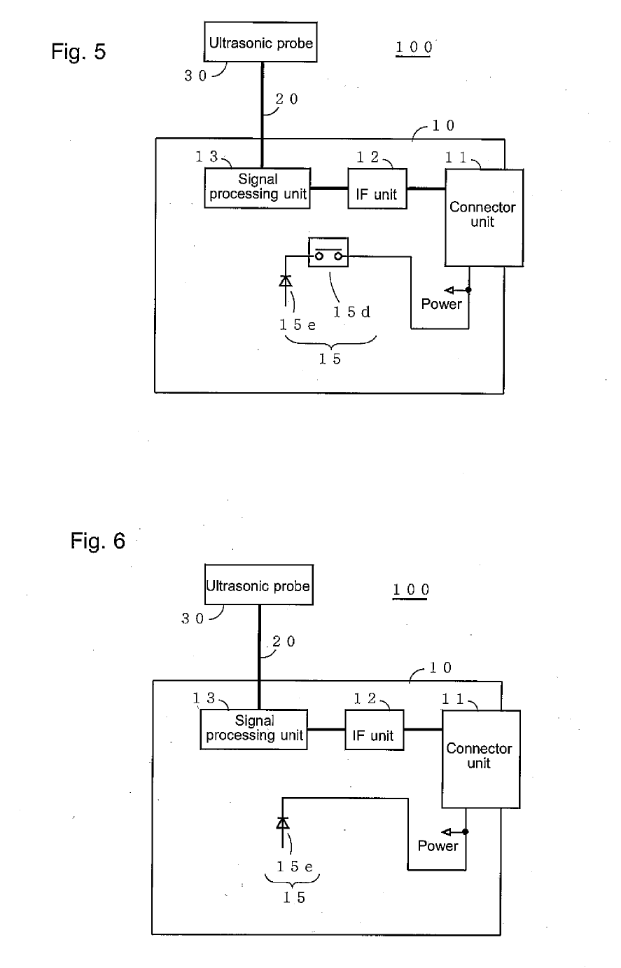

It is also conceivable to dispose an LED 15e inside a translucent name plate as shown in FIG. 5 and to cause the LED 15e to be lit if power is supplied from the ultrasonic diagnosing apparatus body 2 via the connector unit 11 and the switch 15d is “on”.

### Embodiment 4

It is also conceivable to dispose the LED 15e inside a translucent name plate as shown in FIG. 6 and to cause the LED 15e to be lit if power is supplied from the ultrasonic diagnosing apparatus body 2 via the connector unit 11.

Many widely different additional embodiments of the invention may be configured without departing from the spirit and the scope of the present invention. It should be understood that the present invention is not limited to the specific embodiments described in the specification, except as defined in the appended claims.

1. An ultrasonic probe apparatus comprising:
a connector box having on a first face a connector unit to be connected to the probe connector unit of an ultrasonic diagnosing apparatus body and having on a second face an indicator unit which is lit when said connector unit is normally connected to the probe connector unit of said ultrasonic diagnosing apparatus body; and
an ultrasonic probe connected to said connector box by a cable.

2. The ultrasonic probe apparatus according to claim 1, wherein said second face is on the reverse side to said first face.

3. The ultrasonic probe apparatus according to claim 1, wherein said indicator unit varies in the lit or unlit state according to whether the ultrasonic probe apparatus is active or sleeping.

4. The ultrasonic probe apparatus according to claim 3, wherein said indicator unit varies in the color of light according to whether the ultrasonic probe apparatus is active or sleeping.

5. The ultrasonic probe apparatus according to claim 3, wherein said indicator unit varies in the luminous intensity of light according to whether the ultrasonic probe apparatus is active or sleeping.

6. The ultrasonic probe apparatus according to claim 3, wherein said indicator unit has separate light sources including one to be lit when the ultrasonic probe apparatus is active and the other to be lit when the ultrasonic probe apparatus is sleeping.

7. The ultrasonic probe apparatus according to claim 1, wherein said indicator unit also serves as a name plate.

8. The ultrasonic probe apparatus according to claim 1, wherein said connector box is provided with a locking mechanism which locks a state in which said connector unit is connected to the probe connector unit of said ultrasonic diagnosing apparatus body, and said indicator unit is not lit in a state in which said lock is undone.

9. An ultrasonic diagnosing apparatus comprising:
an ultrasonic diagnosing apparatus body having a probe connector unit;
a connector box having on a first face a connector unit to be connected to said probe connector unit and having on a second face an indicator unit which is lit when said connector unit is normally connected to the probe connector unit of said ultrasonic diagnosing apparatus body; and
an ultrasonic probe connected to said connector box by a cable.

10. The ultrasonic diagnosing apparatus according to claim 9, wherein said second face is on the reverse side to said first face.

11. The ultrasonic diagnosing apparatus according to claim 9, wherein said indicator unit varies in the lit or unlit state according to whether the ultrasonic probe apparatus is active or sleeping.

12. The ultrasonic diagnosing apparatus according to claim 11, wherein said indicator unit varies in the color of light according to whether the ultrasonic probe apparatus is active or sleeping.

13. The ultrasonic diagnosing apparatus according to claim 11, wherein said indicator unit varies in the luminous intensity of light according to whether the ultrasonic probe apparatus is active or sleeping.

14. The ultrasonic diagnosing apparatus according to claim 11, wherein said indicator unit has separate light sources including one to be lit when the ultrasonic probe apparatus is active and the other to be lit when the ultrasonic probe apparatus is sleeping.

15. The ultrasonic diagnosing apparatus according to claim 9, wherein said indicator unit also serves as a name plate.

16. The ultrasonic diagnosing apparatus according to claim 9, wherein said connector box is provided with a locking mechanism which locks a state in which said connector unit is connected to the probe connector unit of said ultrasonic diagnosing apparatus body, and said indicator unit is not lit in a state in which said lock is undone.
